(19) United States
(12) Patent Application Publication
(10) Pub. No.: US 2006/0111698 A1
(43) Pub. Date: May 25, 2006

(54) Apparatus and method for performing laser-assisted vascular anastomoses

(76) Inventor: Kihong Kwon, Fullerton, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/994,901

(22) Filed: Nov. 22, 2004

(51) Int. Cl. A61B 18/18

(52) U.S. Cl. 606/8

(57) ABSTRACT

Methods and devices for creating vascular anastomoses are disclosed. In a preferred embodiment, a vein is tissue welded to an artery at a desired anastomosis site. A laser is then used to vaporize tissue within the anastomosis site to form an access pathway between the vein and artery.

### BACKGROUND OF THE INVENTION

1. Field of the Invention

The present invention generally relates to medical devices and methods for welding biological tissue. In particular, the invention relates to performing an anastomosis between body structures. One application involves performing a side-to-side anastomosis of blood vessels during coronary bypass procedures, such as beating heart bypass procedures.

2. Description of the Related Art

A wide variety of medical procedures involve creating an anastomosis to establish fluid communication between two tubular conduits or organs in a patient. Coronary artery bypass graft (CABG) surgery, for example, often involves creating an anastomosis between blood vessels or between a blood vessel and a vascular graft to create or restore a blood flow path to the heart muscles. Such CABG surgery is necessary to overcome coronary artery disease, wherein plaque build-up on the inner walls of the coronary arteries causes narrowing or complete closure of these arteries. This results in insufficient blood flow and deprives the heart muscle of oxygen and nutrients, leading to ischemia, possible myocardial infarction, and even death. CABG surgery may be performed via a traditional open-chest procedure or a closed-chest or port-access thoracoscopic procedure.

CABG surgery may require the creation of one or more anastomosis depending upon whether a “free graft” or a “pedicle graft” is employed. A “free graft” is a length of conduit having open proximal and distal ends. A proximal anastomosis is required to connect the proximal end of the graft to a source of blood (e.g. the aorta) and a distal anastomosis is required to connect the distal end of the graft to the target vessel (e.g. a coronary artery). Free grafts may be autologous, such as by harvesting a saphenous vein or other venous or arterial conduit from elsewhere in the body, or an artificial conduit, such as Dacron® (polyethylene terephthalic ester or PETE) or Goretex® (polytetrafluoroethene or PTFE) tubing. A “pedicle graft” is the result of rerouting a less essential artery, such as the internal mammary artery, from it native location so that it may be connected to the coronary artery downstream of the blockage. The proximal end of the graft vessel remains attached in its native position and only one anastomosis is required to connect the distal end of the graft vessel to the target vessel. In either case, the anastomosis may be between the end of the graft and an aperture in the side wall of the source or target vessel (a so-called “end-to-side” anastomosis) or the anastomosis may be between an aperture in the side wall of the graft and an aperture in the side wall of the source or target vessel (a so-called “side-to-side” anastomosis).

Notwithstanding the foregoing, there remains a need for improved methods and devices for treating obstructive sleep apnea.

### SUMMARY OF THE INVENTION

In one embodiment, a method of treating a patient is provided, comprising the steps of tissue welding the external surface of a first tubular organ to the external surface of a second tubular organ at an anastomosis site in a side-to-side fashion, and creating an access pathway using a laser between the lumen of the first tubular organ and the lumen of the second tubular organ generally through the joining site. In some embodiments, the tissue welding step is performed using UV light. The UV light may be from a laser. In other embodiments, the tissue welding may be performed using any light from a laser. In some instances, the light may be applied externally or intralumenally. The tissue welding step may also be performed using a soldering material. The soldering material may comprise a chromophore, a biological soldering material, or combination thereof. In one embodiment, the biological soldering material is selected from a group consisting of fibrinogen, albumin, myoglobin, elastin and collagen, or combination thereof. In one embodiment, the creating step is performed with a laser positioned within the first tubular organ. The laser may be an excimer laser, a CO2 laser, a YAG laser or any other laser known in the art. In some embodiments, the method further comprises the step of dilating the second tubular organ at least about the joining site. The dilating step may be performed before or during the creating step. The dilating step may also be performed by administering dilating agent into the second tubular organ. The dilating agent may be nitroglycerin or papaverine. In some instances, the dilating step may be performed by administering dilating agent onto the external surface of the second tubular organ, depending upon the particular dilating agent used. In another embodiment, the dilating step may be performed by compression of the second tubular organ adjacent to the joining site. In some instances, the dilating step is performed by compression of the second tubular organ downstream from the joining site with respect to the blood flow in the second tubular organ. The method may also further comprise the step of inserting a protection catheter into the second tubular organ.

In one embodiment, a kit or system for performing vascular anastomoses is provided, comprising a tissue welding system and a laser configured to create an opening between two sealed tubular organs. In some embodiments, the kit or system of the tissue welding system comprises a biological welding agent. In a further embodiment, the tissue welding system further comprises a light source for activating the biological welding agent. In other embodiments, the tissue welding system comprises a chromophore and a light source for activating the chromophore.

Further features and advantages of the present invention will become apparent to those of skill in the art in view of the disclosure herein, when considered together with the attached drawings and claims.

### BRIEF DESCRIPTION OF THE DRAWINGS

The structure and method of using the invention will be better understood with the following detailed description of embodiments of the invention, along with the accompanying illustrations, in which:

FIGS. 1A through 1E depict one embodiment of the invention where a venous graft is anastomosed to an artery.

FIGS. 2A through 2C depict one embodiment of the invention where a graft is attached to a vessel with two anastomosis sites.

FIG. 3 depicts one embodiment of the invention utilizing a end-emitting laser catheter.

FIG. 4 depicts one embodiment of the invention utilizing manual compression to cause dilation of the anastomosis site during use of a laser to create the access pathway.

FIGS. 5A and 5B depict one embodiment of the invention comprising an injection of a vessel dilating agent prior to use of a laser to create the access pathway.

FIG. 6 depicts one embodiment comprising a laser protection catheter during use of a laser to create the access pathway.

### DETAILED DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be routine undertaking for those of ordinary skill in the art having the benefit of this disclosure. It is furthermore to be readily understood that, although discussed below primarily within the context of coronary artery bypass graft surgery (CABG), the anastomosis system of the present invention may be employed in any number of applications and/or procedures wherein it is desired to establish fluid communication between two conduits, including the peripheral vascular system, urinary tract, gastrointestinal system, lymphatic system and other organ systems. The anastomosis system and method disclosed herein boasts a variety of inventive features and attributes that warrant patent protection, both individually and in combination.

Current methods available for creating an anastomosis include hand suturing of the vessels together. Connection of interrupted vessels with stitches has inherent drawbacks. For example, it is difficult to perform and requires great skill and experience on the part of the surgeon due in large part to the extremely small scale of the vessels. Coronary arteries typically have a diameter in the range of between about 1 to about 5 mm, and the graft vessels have a diameter on the order of about 1 to about 4 mm for an arterial graft such as a mammary artery, or about 4 to about 8 mm for a vein graft such as a saphenous vein. In closed-chest or port access procedures, the task of suturing is even more challenging due to the use of elongated instruments positioned through the access ports for approximating the tissues and for holding and manipulating the needles and sutures used to make the anastomoses. Sutures also cause additional tissue damage during their placement and tying, and also result in the introduction of a foreign material into the body, increasing the risk for further damage or rejection. Moreover, sutures do not necessarily result in a water tight seal and may require a long healing time. Other drawbacks of connection with sutures are the long duration of the operation, during which period in conventional open-heart CABG surgery the heart is arrested and the patient is maintained under cardioplegic arrest and cardiopulmonary bypass. Cardiopulmonary bypass has been shown to be the cause of many of the complications that have been reported in conventional CABG, such as stroke. The period of cardiopulmonary bypass should be minimized, if not avoided altogether, to reduce patient morbidity.

One approach to coronary artery bypass grafting that avoids cardiopulmonary bypass is to perform the suturing procedure on a beating heart. Studies have shown that performing CABG without cardiopulmonary bypass and cardioplegic arrest may result in reduced risk of myocardial injury, systemic inflammatory response, renal and neurological dysfunction. (Ngaage DL, Off-pump coronary artery bypass grafting: simple concept but potentially sublime scientific value, Med Sci Monit. 2004 Mar;10(3):RA47-54) At present, however, an anastomosis between a stenotic coronary artery and a bypass graft vessel during beating heart bypass is technically more demanding and presents numerous obstacles, given the continuous cardiac translational motion which makes meticulous microsurgical placement of graft sutures extremely difficult. The constant translational motion of the heart and bleeding from the opening in the coronary artery hinder precise suture placement in the often tiny coronary vessel.

The drawbacks of hand suturing have led to the development of various approaches to performing sutureless vascular anastomoses. These include the use of rigid rings in U.S. Pat. No. 4,624,257 to Berggren et al., stapling devices in U.S. Pat. No. 4,350,160 to Kolesov, et al., anastomotic fittings in U.S. Pat. No. 4,366,819 to Kaster. These anastomotic devices, however, continue to exhibit problems similar to those associated with sutured anastomoses, such as fistulas, granulomas, and neuromas caused by tissue incompatibility, as well as leakage problems.

Tissue welding is a procedure of using light energy to bond tissues together. Although the mechanisms of the tissue welding process are not yet completely understood in the case of vascular tissue, it is surmised that the light acting on the tissue leads to a coagulation of proteins and thus to an anastomotic joining of the biological surfaces. The light source used for tissue welding is preferably but not necessarily a laser light source. Laser soldering is a method of improved tissue welding by introducing a proteinaceous solder material between the tissues or other surfaces to be joined prior to exposure to the laser. The solder material used may include but is not limited to fibrinogen, albumin, myoglobin, elastin and collagen. U.S. Pat. No. 5,152,759 to Parel, et al., U.S. Pat. No. 6,323,037 to Lauto, et al., and U.S. Pat. No. 7,607,522 to Hamblin et al., herein incorporated by reference in their entirety, describe other solder compositions that may be used for tissue welding. Soldering is beneficial for its ability to enhance bond strength, lessen collateral damage, and enlarge the parameter window for a successful bond. The solder is able to do this by holding the tissues together, creating a larger bonding surface area, sometimes by as much as two degrees of magnitude. In addition, the proteinaceous solder material may be mixed with a chromophore or light absorber, to interface with the applied laser light into the solder and release the laser energy. Chromophores have also been used alone for laser tissue welding. The chromophore may be selected by those skilled in the art to have a maximum absorption wavelength tailored to the wavelength of the laser light used to perform the laser soldering. Chromophores that have been used include but are not limited to indocyanine green with 805 nm diode lasers, flouroscein with 532 nm frequency-doubled Nd:YAG lasers, and chlorinₑ₆ with argon lasers.

Laser tissue welding has been used successfully in nerve, skin, and arterial applications. The technique offers significant advantages for securing and sealing skin grafts, repairing solid-tissue organ damage, minimizing laceration trauma, and closing surgical incisions. A major advantage of tissue welding is the instant tissue healing and sealing that it offers, which allows for a quicker return to functional recovery.

Tissue welding technology has been used with lasers emitting a variety of wavelengths, including infrared and ultraviolet laser sources. Lasers that may be used for tissue welding or soldering include but are not limited to excimer, argon, KTP (potassium-titanyl-phosphate), pulsed dye, ruby, alexandrite, diode, Nd:YAG, Ho:YAG, Er:YAG and CO₂ lasers. One skilled in the art can select a particular laser for use with the invention depending on the particular anatomical considerations, soldering agent and other factors.

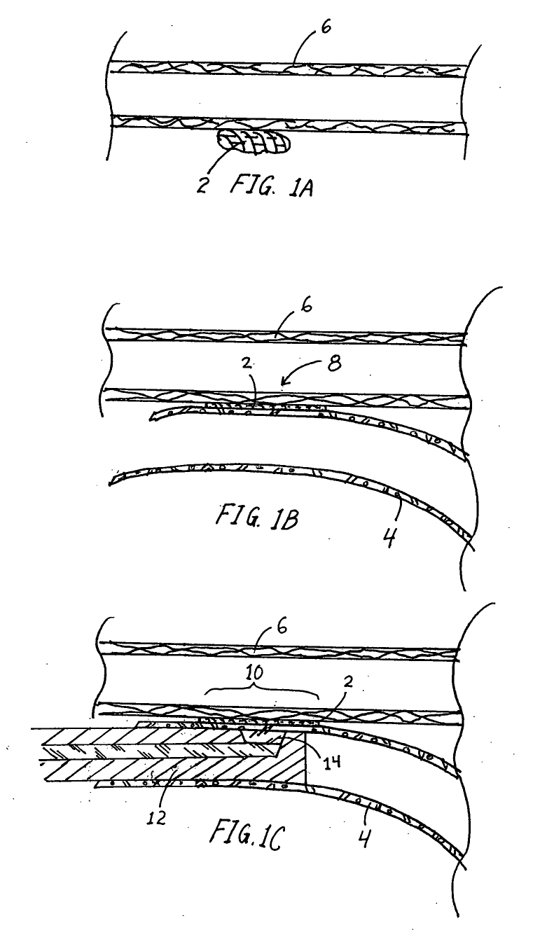

In one preferred embodiment, the invention comprises a method for performing an anastomosis of a venous graft to a coronary artery. Referring to FIG. 1A and 1B, a biological soldering agent 2 is applied between the venous graft 4 and coronary artery 6 at a desired first anastomosis site 8. In one embodiment, the venous graft 4 and coronary artery 6 are generally oriented in a side-to-side relationship with the longitudinal axes of the venous graft 4 and coronary artery 6 arranged in parallel fashion in order to reduce flow disturbances through the anastomosis site 8. In other embodiments, the venous graft 4 and coronary artery 6 are arranged within the range of about 0 degrees to about 180 degrees with respect to the longitudinal axis of the coronary artery in a plane generally tangential to the surface of the heart muscle at the anastomosis site. The surface area of the anastomosis site 8 between the vein graft 4 and coronary artery 6 may be about 0.25 cm² to about 4 cm², preferably about 0.50 cm² to about 3 cm², and sometimes about 0.50 cm² to about 3 cm². In some embodiments, UV light is applied externally to the vein graft 4 and artery 6 to cause tissue welding and tissue sealing at the anastomosis site 8 to form a sealed zone 10. As shown in FIG. 1C, an excimer laser 12 or other laser is inserted into the venous graft 4 and oriented until the laser output port 14 overlies the sealed zone 10. Preferably, a laser catheter with a side-projecting port is used, but this is not required. As illustrated in FIG. 1D, the laser 12 is activated to remove or vaporize a portion of the tissue within the sealed zone 10 such that a conduit or access pathway 16 is created through the sealed zone 10 while leaving at least a rim or perimeter 18 of sealed zone 10 around the access pathway 16. The cross sectional shape of the access pathway 16 may be any of a variety of closed plane shapes, including but not limited to a circle, ellipse, square, or rectangle. The access pathway 16 may also comprise a straight or curved slit, or combination thereof, within the first anastomosis zone. Preferably, the access pathway 16 has an elongated shape oriented with respect to the artery in order to reduce possible flow disturbances through the anastomosis site. In some embodiments, an oval shaped access pathway 16 is preferred. Referring to FIG. 1E, the end 20 or ends of the venous graft 4 may be closed using conventional suturing, stapling or laser welding as is known in the art. Although the embodiment described above utilizes a venous graft 4, the same procedure may be used to attach an arterial graft, such as an internal mammary artery, to the coronary artery. The invention may also be adapted to create AV grafts in the peripheral vascular system for use with dialysis machines.

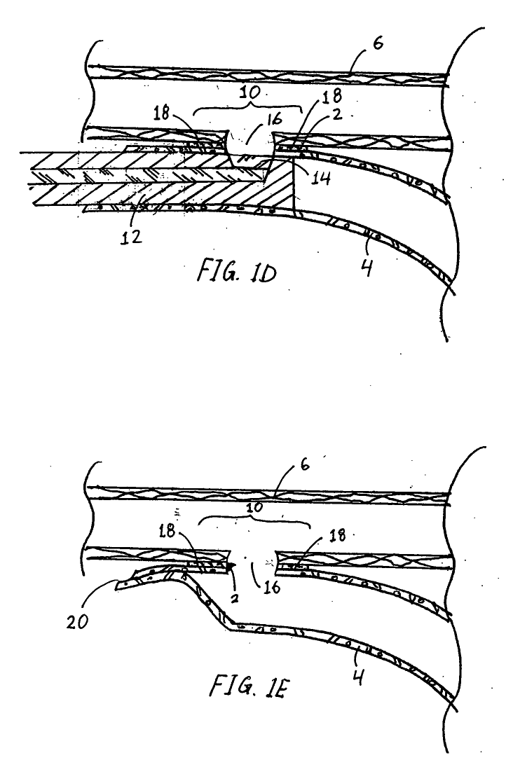

Referring to FIGS. 2A through 2C, in one embodiment, a second anastomosis site and second sealed zone 24 is formed between the venous graft 4 and the coronary artery 6. This may be performed when there is a coronary lesion 26 that cannot be treated by coronary stenting. This artery may be the same or different artery from the one comprising the first sealed zone 10. In some instances, as shown in FIG. 2A, the second sealed zone 24 is formed before the use of the laser 12 to create the first access pathway. The laser is oriented over the second sealed zone 24 to create a second access pathway 28 through the second sealed zone while leaving at least a rim or perimeter of sealed second sealed zone 24 around the second access pathway 28. In other embodiments, the first access pathway 16 is formed before the second sealed zone 24 is formed. In some embodiments, at least one end 30 of the venous graft 4 is closed before either access pathway is created.

In one embodiment, the invention comprises a method for performing a laser-assisted anastomosis of a first tubular organ and a second tubular organ. A tubular organ may include a blood vessel, lymphatic duct, intestine, esophagus, stomach, biliary tree, gall bladder, pancreatic duct, heart, airway, ureter or other tubular organ. A biological agent is applied between the first and second tubular organs at the desired anastomosis site and the tubular organs are sealed. The biological agent may be a proteinaceous soldering material, a lipid soldering agent, a chromophore or any of a variety of biological joining agents known in the art. The joining of the two tubular organs with the biological agent may or may not include laser or light-assisted tissue welding of the two surfaces. The surface area of the anastomosis site can be selected by one skilled in the art and will depend upon the type of tubular organs that are anastomosed, estimated flow of material at the anastomosis site, fluid pressure, if any, and other factors. The light may be applied externally to the external surfaces of the tubular organs, or internally from one or more lumens of the tubular organs. Preferably, ultraviolet light or an UV laser is used to join the surfaces. An access pathway is then created through the two tissues at the sealed anastomosis site using a laser to remove or vaporize at least some of the tissue material within the anastomosis site. Typically, the laser is an excimer laser capable of vaporizing the tissue of the anastomosed organs, but other lasers may also be used. The access pathway may be a linear or curved slit, a circular or oval opening, a square or rectangular opening, a combination thereof, or any other closed shaped opening. In the preferred embodiment, the access is asymmetrical and has a greater dimension with respect to the longitudinal axis of either the artery, graft or an axis therebetween.

In another preferred embodiment of the invention, two tissue planes are anastomosed using a laser. In one embodiment, at least one tissue plane comprises the wall of an artery. In another embodiment, at least one tissue plane comprises the wall of a vein. A biological agent is applied between the two tissue planes at a desired anastomosis site, forming a sealed region. The biological agent may be a bioglue or tissue soldering agent such as a proteinaceous soldering material, a lipid soldering agent, a chromophore, a combination thereof or any of a variety of biological joining agents known in the art. The joining of the two tissue planes may or not include the application of light to enhance the tissue soldering. In some embodiments, the light has a wavelength in the infrared wavelength range. In other embodiments, the light has a wavelength in the ultraviolet wavelength range. In some embodiments, the light emitted is from a laser source. A laser source, which may or may not be separate from the laser source, if any, used for tissue welding, is then inserted against one of the two tissue planes and oriented within the sealed region. The laser source is activated and an access pathway is created within the sealed region.

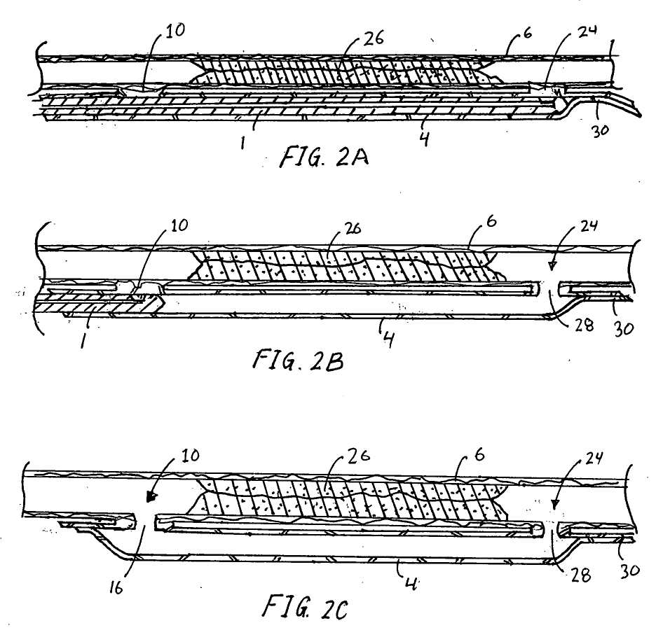

Although the lasers depicted in FIGS. 1C, 1D, 2A and 2B are side-firing laser catheters such as those disclosed in U.S. Pat. No. 6,029,671 to Stevens et al., an end-firing laser 32 may also be used, as shown in FIG. 3. The laser 12, 32 may also be configured with a short depth of focus to provide beam divergence beyond the expected target tissue distance and thereby reduce the potential damage to the posterior or distal wall of the underlying vessel. In some embodiments, the laser 12, 32 has a depth of focus generally about the contact point of the catheter to the target tissue. In some embodiments of the invention, the laser emission is spaced at least 1 mm from the outer surface of the catheter to reduce effects of spherical aberration. In other embodiments, the laser 12, 32 has a focal point about 1 mm to about 3 mm or more from the surface of the laser device.

In some embodiments of the invention, the portions of the laser catheter 12 proximal to the firing port may have indicators to allow the operator to align and orient the laser firing port with respect to the sealed zone. In one embodiment, the indicators are calibrated for creating a sealed zone within a certain distance from the end of the vessel in which the laser catheter is inserted. Other landmarks may also be used, including those on the heart itself. These indicators may include markings to indicate the positioning of the catheter along the longitudinal axis of the catheter and/or the rotational positioning of the catheter about its longitudinal axis. These indicators may also be radio-opaque to allow visualization of the catheter positioning on x-ray imaging or fluoroscopy. In another embodiment, a separate set of radio-opaque indicators are provided on the catheter. In still another embodiment, only the radio-opaque indicators are provided.

In some embodiments, the tissue about the anastomosis site is cooled to reduce undesired tissue damage from the use of a vaporizing laser. In one embodiment, the tissue is cooled by applying a cooling probe against the tissue about the anastomosis site. In one embodiment, the cooling probe may be integrated with a laser catheter used to create the access within the sealed zone. In another embodiment, a cryogen is sprayed to cool the tissue. In still another embodiment, a cooled biocompatible liquid is injected about the tissue or into the lumen about the tissue. Tissue cooling may be performed before, during and/or after the application of the laser.

In some embodiments of the invention, it is hypothesized that the posterior wall of the coronary artery is not subject to a clinically significant damage from the laser used to create the access pathway because the flow of blood may act as a continuous heat sink to prevent damage to the posterior wall, but no embodiment is limited to this hypothesis. This protection may depend upon the power and wavelength of the laser used to create the access pathway and the wavelength absorption spectrum of the blood, red blood cells and/or hemoglobin as well as the cardiac output of the patient. In some embodiments of the invention, light from a CO₂ laser or Er:YAG laser, which is strongly absorbed by water in the blood, may be preferred. In other embodiments, an argon laser or pulsed dye laser which is strongly absorbed by hemoglobin in the blood is preferred.

In other embodiments, protection of the posterior wall of the artery may be desirable. To protect the posterior or distal inner wall of the artery from damage during the creation of the access at the sealed zone, the laser may be configured to a depth of focus at the contact point of the catheter with the lumen or a very short distance thereafter and immediately diverge to reduce clinically significant damage to the posterior wall of the artery.

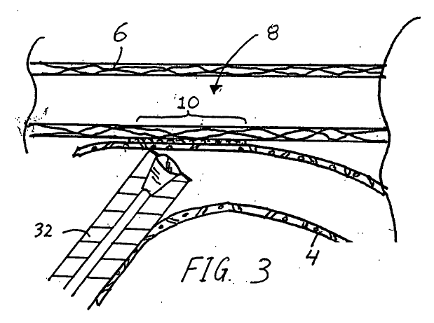

In another embodiment of the invention, depicted in FIG. 4, the artery 6 or underlying blood vessel is compressed at a occlusion site 34 to the anastomosis site. This causes distention of the artery 6 proximal to the occlusion site 34 and will increase the distance between the proximal vessel wall 36 comprising the anastomosis site 8 and the opposing inner vessel wall 38. This increased distance may further reduce any potential damage from the laser 12.

In still another embodiment, the artery or underlying vessel is occluded at both a distal site and proximal site to the anastomosis site. A biocompatible fluid, such as saline, may be injected in the unoccluded artery between the two occlusion sites to distend the artery. In some embodiments, the biocompatible fluid may have a particular wavelength absorption characteristic that may absorb the wavelength of the penetrating laser and reduce the risk of damage to the posterior wall of the artery.

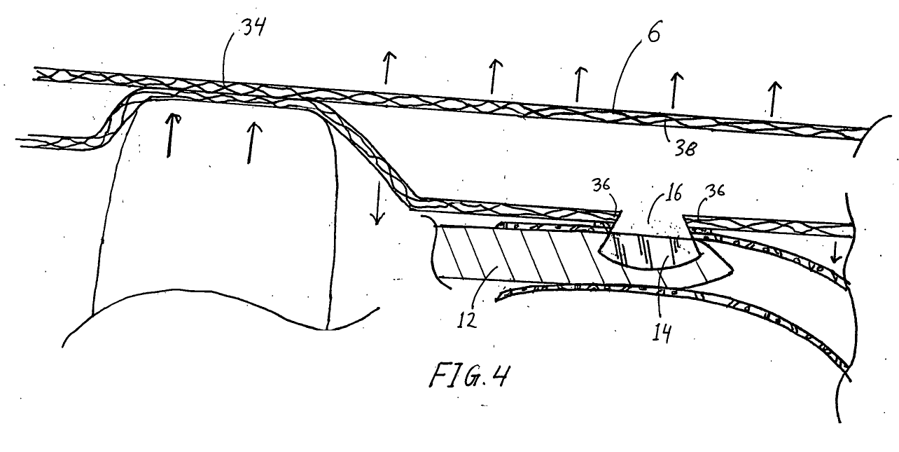

In addition to mechanical distention of the vessel at the anastomosis site, pharmaceutical dilation or distention of the blood vessel may also be performed using a dilating agent such as nitroglycerin or papaverine. Referring to FIG. 5A, a needle 40 may be inserted into the underlying blood vessel 42 having a diameter d′ from the overlying blood vessel 44 and a locally acting pharmaceutical agent, such as nitroglycerin, may be injected into the underlying artery 42 to cause dilation to a larger diameter d″, as shown in FIG. 5B. In one embodiment, papaverine is preferred as the dilating agent because it can be topically applied onto the underlying blood vessel and does not require intravenous injection.

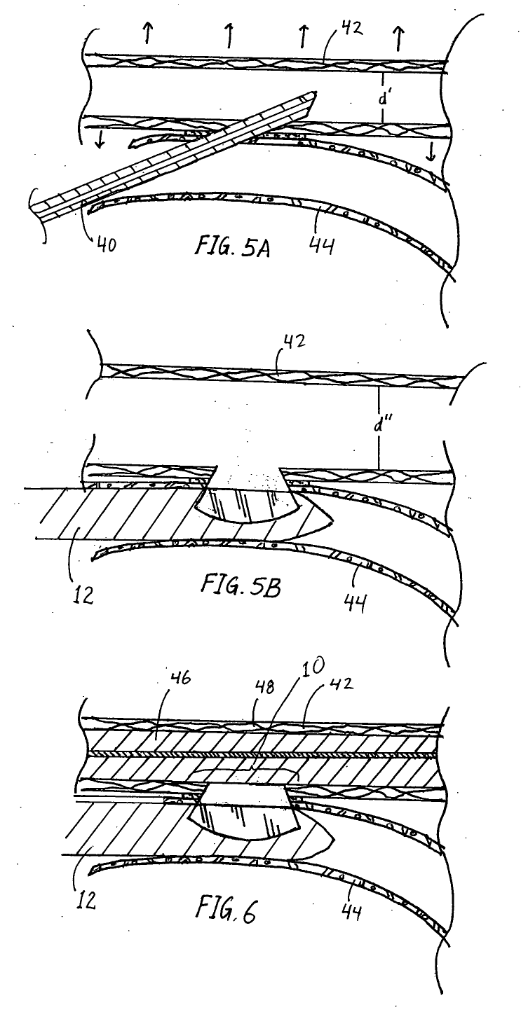

In another embodiment, illustrated in FIG. 6, a protection catheter 46 is inserted into the underlying blood vessel 42 to protect the distal blood vessel wall 48 once the laser 12 has penetrated through the sealed zone 10. The protection catheter 46 is designed to absorb or diffuse the laser beam upon penetration through the sealed zone 10. Typically, the protection catheter 46 is inserted from a peripheral vascular access site such as the right femoral artery and then maneuvered to the anastomosis site. However, insertion of the protection catheter 46 is not limited to peripheral vascular sites and may also be inserted at a. central blood vessel site. The protection catheter 46 may also comprise a distal emboli protection system to retain any emboli or vessel wall remnants that may flow downstream from the anastomosis site 8. In another embodiment, the protection catheter 46 further comprises a sensor capable of detecting the penetration of the laser through the vessel wall. In some instances, the sensor may be coupled to a control unit that can control shut off the laser 12 upon vessel wall penetration.

While this invention has been particularly shown and described with references to embodiments thereof, it will be understood by those skilled in the art that various changes in form and details may be made therein without departing from the scope of the invention. For all of the embodiments described above, the steps of the methods need not be performed sequentially.

What is claimed is:

1. A method of treating a patient, comprising the steps of:
tissue welding the external surface of a first tubular organ to the external surface of a second tubular organ at an anastomosis site in a side-to-side fashion; and
creating an access pathway using a laser between the lumen of the first tubular organ and the lumen of the second tubular organ generally through the joining site.

2. The method of treating a patient as in claim 1, wherein the tissue welding step is performed using UV light.

3. The method of treating a patient as in claim 2, wherein the tissue welding step is performed using UV light from a laser.

4. The method of treating a patient as in claim 1, wherein the tissue welding step is performed using light from a laser.

5. The method of treating a patient as in claim 1, wherein the light of the tissue welding step is applied externally.

6. The method of treating a patient as in claim 1, wherein the light of the tissue welding step is applied intralumenally.

7. The method of treating a patient as in claim 1, wherein the tissue welding step is performed using a soldering material.

8. The method of treating a patient as in claim 7, wherein the soldering material of the tissue welding step comprises a chromophore.

9. The method of treating a patient as in claim 8, wherein the soldering material of the tissue welding step further comprises a biological soldering material.

10. The method of treating a patient as in claim 7, wherein the soldering material of the tissue welding step comprises a biological soldering material.

11. The method of treating a patient as in claim 10, wherein the biological soldering material of the tissue welding step is selected from a group consisting of fibrinogen, albumin, myoglobin, elastin and collagen, or combination thereof.

12. The method of treating a patient as in claim 1, wherein the creating step is performed with a laser positioned within the first tubular organ.

13. The method of treating a patient as in claim 12, wherein the laser of the creating step is an excimer laser.

14. The method of treating a patient as in claim 12, wherein the laser of the creating step is a CO₂ laser.

15. The method of treating a patient as in claim 12, wherein the laser of the creating step is a YAG laser.

16. The method of treating a patient as in claim 1, further comprising the step of:
dilating the second tubular organ at least about the joining site.

17. The method of treating a patient as in claim 16, wherein the dilating step is performed before or during the creating step.

18. The method of treating a patient as in claim 16, wherein the dilating step is performed by administering dilating agent into the second tubular organ.

19. The method of treating a patient as in claim 18, wherein the dilating agent is nitroglycerin.

20. The method of treating a patient as in claim 16, wherein the dilating step is performed by administering dilating agent onto the external surface of the second tubular organ.

21. The method of treating a patient as in claim 20, wherein the dilating agent of the dilating step is papaverine.

22. The method of treating a patient as in claim 16, wherein the dilating step is performed by compressing the second tubular organ adjacent to the joining site.

23. The method of treating a patient as in claim 22, wherein the dilating step is performed by compressing the second tubular organ downstream from the joining site with respect to the blood flow in the second tubular organ.

24. The method of treating a patient as in claim 1, further comprising the step of inserting a protection catheter into the second tubular organ.

25. A kit or system for performing vascular anastomoses, comprising:
a tissue welding system; and
a laser configured to create an opening between two sealed tubular organs.

26. The kit or system of claim 25, wherein the tissue welding system comprises a biological welding agent.

27. The kit or system of claim 26, wherein the tissue welding system further comprises a light source for activating the biological welding agent.

28. The kit or system of claim 25, wherein the tissue welding system comprises a chromophore and a light source for activating the chromophore.
